(19) United States
(12) Patent Application Publication
(10) Pub. No.: US 2003/0215506 A1
(43) Pub. Date: Nov. 20, 2003

(54) Methods and compositions for enhancement of creatine transport

(76) Inventor: Eric H Kuhrts, Bodega, CA (US)

Correspondence Address: ERIC H. KUHRTS, P.O. Box 16, Bodega, CA, 94922, US

(21) Appl. No.: 10/153,495

(22) Filed: May 17, 2002

(51) Int. Cl. A61K 38/30; A61K 9/22; A61K 35/20; A61K 31/198

(52) U.S. Cl. 424/468; 424/535; 514/12; 514/565

(57) ABSTRACT

Disclosed are creatine transport enhancing formulations of creatine with an IGF-1 modulating agent such as whey protein, colostrum, or recombinant IGF-1. The creatine and IGF-1 modulating substance are preferably in sustained-release form so as to modulate and facilitate the absorption and transport of the creatine to muscle. The sustained-release combination of creatine monohydrate and IGF-1 modulating substance enhance the supply of creatine to the muscles especially during extended athletic activity or body building. The whey protein is preferably a whey protein isolate or concentrate that contains a higher percentage of biologically active molecules such as immunoglobuline A or IGF-1. The colostrum is also preferably one containing a high percentage of IgG, such as colostrum containing from 10-40% IgG.

### BACKGROUND OF THE INVENTION

This invention relates to the enhancement of creatine transport to muscle. More particularly, pharmaceutical compositions containing whey protein or colostrum or immunoglobulins such as IgG, or insulin like growth factor (IGF-1) are combined with creatine, either in sustained-release or immediate-release form, to enhance the activity and transport of creatine in skeletal muscle. The compositions are useful for building muscle mass, and prolong and increase the activity and supply of creatine to and in muscle tissue.

### SUMMARY

In one embodiment, the invention is directed to a creatine transport enhancing composition comprising creatine or a pharmaceutically acceptable salt, ester, polypeptide, complex, prodrug, metabolite, or derivative of creatine; and one or more of an IGF-1 mediating substance or insulin mediating substance. The composition may be delivered orally. The creatine includes “creatine-like” compounds such as creatine monohydrate, carnitine creatinate, creatine pyruvate, or cyclocreatine. In a preferred embodiment, the IGF-1 mediating substance or insulin mediating substance is whey protein, colostrum, or immunoglobilins such as IgG. In exemplary embodiments, the creatine is creatine monohydrate in an amount between about 500 mg and about 10 grams and the IGF-1 mediating substance is in an amount between about 500 mg and about 30 grams. The IGF-1 mediating substance is preferably isolated whey protein or colostrum that has been microencapsulated to protect the large biologically active macromolecules from being completely destroyed in the harsh environment of the stomach. In an exemplary embodiment, the IGF-1 mediating substance is sufficient to elicit a significant increase in blood flow to the extremities and muscles.

The invention further includes a method of enhancing creatine transport to muscle in a mammal comprising the steps of administration of creatine or a pharmaceutically acceptable salt, ester, polypeptide, complex, prodrug, metabolite, or derivative of creatine; and coadministration of one or more of an IGF-1 mediating substance. In a preferred embodiment, the IGF-1 mediating substance or insulin mediating substance is microencapsulated whey protein isolate or colostrum. In one embodiment, the creatine is creatine monohydrate and the creatine is also sustained-release. In another embodiment, the creatine may be immediate-release and the IGF-1 mediating substance is sustained-release.

The invention further includes a method of increasing muscle mass in comprising the steps of administration of creatine or a pharmaceutically acceptable salt, ester, polypeptide, complex, prodrug, metabolite, or derivative of creatine; and coadministration of an IGF-1 mediating substance such as whey protein or colostrum. In an exemplary embodiment of this method, the creatine of the composition is creatine monohydrate in a sustained released form in an amount between about 500 mg and about 10 grams and the IGF-1 mediating substance is whey protein isolate in a sustained released form in an amount between about 500 mg and about 30 grams.

In preferred embodiments, one or more substances of the composition are in sustained-release form. For example, creatine monohydrate and the IGF-1 modulating substance are sustained-release. In exemplary embodiments the sustained-release agent comprises algal polysaccharides, chitosan, pectin, glucomannan, guar gum, xanthan gum, gum arabic, gum karaya, locust bean gum, keratin, laminaran, carrageenan, cellulose, modified cellulosic substances such as cellulose ether derivatives; methylcellulose, hydroxypropylmethylcellulose, hydroxypropylcellulose, hydroxyethylcellulose, sodiumcarboxymethylcellulose, carboxymethylcellulose carboxypolymethylene, acrylic resin polymers, polyacrylic acid and homologues, polyethylene glycol, polyethylene oxide, polyhydroxylalkyl methacrylate, polyvinylpyrollidine, polyacrylamide, agar, zein, stearic acid, high melting point oils, waxes, gelatin, or combinations thereof. In an exemplary embodiment the sustained-release particles are produced by coating with an oil with a melting point greater than 100° F. In a preferred embodiment, the oil is a hydrogenated soy oil with a melting point of approximately 150° F.

In one exemplary embodiment the amount of creatine is at least 500 mg. and the whey protein isolate is 20 grams. In another embodiment, the creatine is creatine monohydrate in a single dose is about 500 mg. to 10 grams, and the IGF-1 mediating agent is colostrum containing immunoglobulins such as IgG or IGF-1 itself. The colostrum can be present as a single dose is 250 mg.−30 grams. In another exemplary embodiment, the amount of creatine monohydrate in a single dose is 5 grams and the amount of colostrum in a single dose is 2 grams. In one embodiment the amount of sustained-release colostrum is 2 grams and the amount of sustained-release creatine monohydrate is 5 grams.

### DESCRIPTION

Substances generated by cells within the vessel wall have been demonstrated to be important regulators of smooth muscle tone. The vascular endothelium is responsible for the generation of these vasoactive compounds.

IGF-1 or insulin-like growth factor, is a polypeptide hormone that has close structural resemblance to insulin, and has been shown to weakly stimulate glucose uptake. IGF-1 is also a stimulator of muscle blood flow, and can significantly augment blood flow to muscle without causing an increase in glucose uptake (Pendergrass, M et. al. Am J. Physiol. 275: E345-E350, 1998).

Creatine, or methyl guanidine-acetic acid is manufactured by the body to supply energy to the muscles. One of the endogenous precursors for the production of creatine is L-arginine. Creatine can be made by the body (endogenous) or be supplied by the diet or through supplementation (exogenous). The liver is responsible for the majority of the endogenous biosynthesis of creatine within the body, as well as the metabolism of exogenous creatine from supplemental dietary creatine or L-arginine.

One of the substances that stimulates insulin lie growth factor-1 (IGF-1) is the amino acid L-arginine (Barbul A, Surgery; 114:155-60, 1993). IGF-1 stimulates sodium-potassium activity, which increases creatine accumulation in a muscle cell line (Odoom, J. E. et al, Mol. Cell. Biochem 158;179-188, 1996). The majority of oral creatine absorption into the blood stream and uptake by skeletal muscle occurs by binding to a specific transport protein. In fact, the transport of most amino acids into the cytoplasm of cells Also involves binding of the amino acid to a carrier protein. This process is sodium dependent and can be saturated. Therefore, transport of most amino acids is facilitated by sodium ions, and a gradient is formed consisting of a high extracellular/intracellular sodium differential, which acts as a channel. By stimulating endogenous production of IGF-1 substances such as whey protein and colostrum are capable of inducing this sodium-potassium activity thereby enhancing creatine transport to and accumulation in muscle. Colostrum and whey protein also contain IGF-1 itself, in addition to IgG (immunoglobulins). The IGF-1 mediating substance may therefore be supplied exogenously or produced in the body (endogenously). Human recombinant IGF-1, such as is available from Genentech, San Francisco, Calif., can also be used as a source of exogenous IGF-1. If human recombinant IGF-1 is used, it is preferably injected along with oral administration of the creatine component.

Infusion of IGF-1 into humans for 4 hours has been shown to stimulate protein synthesis in skeletal muscle (Fryburg, D. A.: Am J Physiol. 267; E331-E336, 1994). Furthermore, IGF-1 stimulates sodium-potassium activity, and increases creatine accumulation in a muscle cell line (Odoom, J. E. et al; Mol Cell Biochem:158; pp179-188, 1996).

After being made in the liver, creatine is transported by the blood and taken up by muscle cells where it is converted into creatine phosphate, otherwise known as phosphocreatine. The enzyme responsible for this conversion is creatine kinase which is found inside muscle cells. Creatine kinase is also found in the liver, and is an enzyme especially susceptible to oxidative degradation. As creatine cycles back and forth between creatine and phosphocreatine, it produces energy to the muscle cells, but only briefly. This immediate burst of energy has an extremely short half life of about 15 seconds. Dietary supplementation with large amounts of creatine, or creatine loading, has been one way of attempting to overcome the quick exhaustion of creatine stores when there is intense and prolonged activation of muscles during athletic activity or weight training. It is believed that by loading the muscles with extra creatine, more creatine would be available for energy production by the muscles after the initial exhaustion of endogenous creatine. But the amount of creatine present in muscle cells can saturate the sodium transport system responsible for enabling more creatine to enter the muscles, reducing the flow of new creatine as already present creatine stores are blocking the diffusion gradient. Consuming more immediate-release creatine does not necessarily push more creatine into the muscles because it can shut down the sodium pump responsible for shuttling the creatine into muscle to begin with. In addition, a high creatine concentration will downregulate muscle creatine transport (Proc. Natl. Acad. Sci. USA 85:807-811, 1988).

Creatine accumulation can be substantially increased in human skeletal muscle when ingested with large quantities of simple carbohydrates, but the amounts of carbohydrates necessary are far too high to be pysiologically acceptable for repeated use. For example, a 94 gram dose of carbohydrates in the form of glucose and simple sugars was needed with each 5 gram dose of creatine to increase muscle creatine by about 25%. (Am J. Physiol. 271 (Endocrinol. Metab. 34) E821-E826, 1996). This effect is believed to be related to carbohydrate-mediated insulin release, which presumably would stimulate sodium-dependent muscle creatine transport. Insulin has been demonstrated to stimulate muscle blood flow, and insulin mediated vasodilation, or perfusion, is driven by endothelium-derived nitric oxide. However, nitric oxide may only act as a mediator for this process. IGF-1 does not necessarily mimic the actions of insulin, particularly concerning lipid metabolism and adipose tissue.

The rate of muscle perfusion or vasodilation is an important determinant of the overall rate of glucose uptake. The rate of infusion of insulin effects the transport and storage of creatine, but the enhancement of creatine transport and storage by an IGF-1 mediating substance such as whey protein or colostrum is unexpected.

The term “creatine analog” is intended to include compounds which are structurally similar to creatine, compounds which are recognized in the art as being analogs of creatine, and/or compounds which share the same or similar function as creatine. Many creatine analogs have been previously synthesized and described in; Rowley et al., J. Am. Chem. Soc. 93:5542-5551 (1971) and other publications. Cyclocreatine, homocyclocreatine, and carbocreatine may also be used. The term “creatine-like compounds” may be used to include creatine, creatine salts, creatine analogs, and creatine analog salts.

By incorporating an IGF-1 mediating substance or a biological equivalent or IGF-1 itself with creatine, muscle creatine stores may be increased. An even more effective formulation for accomplishing creatine muscle transport and accumulation is the combination of an oral sustained-release formulation of the IGF-1 mediating substance with sustained-release creatine. Sustained-release or microencapsulated macromolecules such as IgG or IGF-1 are more effective because more of the large molecules remain intact and survive the harsh environment of the stomach, and are therefore biologically available in the small intestine and the large intestine to mediate IGF-1. Furthermore, by coupling the slow presentation of both substances simultaneously, a type of shuttle for more effective delivery of creatine to muscle is provided.

By slowing down the rate of presentation of the creatine to the liver and the muscles, especially during intense exercise or body building workouts, the need for normal creatine loading, which is inefficient, is avoided. Instead, the supply of creatine is constant, and is not working against a concentration gradient for entry to muscle. The slow, long term supply of creatine, which spans many hours of exercise activity, provides a metered injection of creatine as it is exhausted from muscle stores. This type of system is more effective during intense muscular activity than during sedentary periods because of the increased catabolism of creatine to creatinine.

Likewise, long term vasodilatory effects from IGF-1 mediation and a sustained increase in blood supply drives creatine and other nutrient and energy rich co-factor availability to skeletal muscle. By increasing the supply and controlling the rate of blood flow to muscle, the rate of glucose uptake can be effected, which should serve to extend and prolong the integrity of creatine kinase, and thereby facilitate the cycling of creatine to phosphocreatine. This process enhances creatine stores in myocytes, and provides a better environment for the entire process.

The enzyme responsible for conversion of creatine to phosphocreatine is creatine kinase, which is found in muscle cells, and the liver. Creatine kinase is especially susceptible to oxidative degradation. As creatine cycles back and forth between creatine and phosphocreatine, it produces energy to the muscle cells. Intense exercise increases production of reactive oxygen species or free radicals, which prevents creatine kinase from producing phosphocreatine, and instead produces the breakdown product creatinine. Increased levels of creatinine and creatine kinase are often a sign of muscle damage. Too much creatine kinase shifts the pathway away from conversion to phosphocreatine towards the end product of creatine catabolism, which is creatinine. In this case, catabolism is the breakdown of the system, whereas metabolism is the conversion to beneficial end products. Catabolism moves the process towards creatinine, which is not beneficial or useful for conversion to muscle mass or energy.

The maximum oxygen uptake or VO2 max, an index for aerobic capacity, is partly determined by the availability and control of blood flow to the active muscles. Aerobic capacity can be increased or reduced by vascular reactivity.

The preferred IGF-1 mediating supplement to be coadministered with creatine would be a bovine source of immunoglobulins or IGF-1 such as whey protein or colostrum. Whey protein and colostrum are products that are readily available from the dairy industry. Whey protein isolate and whey protein concentrate are derived from whey protein, which in turn is a milk byproduct. Likewise, colostrum is also derived form lactating cows as a byproduct of the dairy indistry. Whey protein isolate can contain up to 2-5% immunoglobulin A (IgG), and colostrum contains an even higher percentage, varying from 15-30% IgG. Oral consumption of these macromolecules is subject to the harsh environmnet of the stomach, which contains acids and enzymes that degrade the macromolecules within 5-10 minutes. Injection or infusion of human recombinant IGF-1 avoids these problems, because the gastrointestinal tract is bypassed.

Enhancing the bioavailability of orally administered peptide and protein drugs has been an ongoing, yet elusive goal of the biotechnology industry for many years. Oral delivery of large molecules is usually less than 1% of a given dose. This has resulted in the need for excessive doses of the drug, which is uneconomical and impracticable, and, in some cases may result in side effects. Even so, some of these molecules are so potent that such small amounts are capable of producing a therapeutic effect. Because of this limitation, most of these drugs, such as insulin, must still be injected, because they are inactivated when taken orally. Since the immunoglobulins and other large molecules that are contained in colostrum are also considered macromolecules, and hence subject to major destruction or degradation in the stomach, the same issues are important for the oral delivery of biologically active macromolecules in colostrum or whey protein as for the complex compounds that are the products of biotechnology, such as human recombinant IGF-1.

Enzymes in the gut provide a formidable barrier to absorption of peptide and protein compounds from the gastrointestinal (GI) tract. These enzymes and the acidic environment of the gut are designed to break these substances down into individual amino acids, or small peptides consisting of two to six amino acid residues prior to the appearance in the portal circulation. Research has now verified that exposure of colostrum to the acids and enzymes in gastric acid secretions results in a substantial loss of activity both in-vitro and in-vivo. In experiments conducted by the inventor, in simulated gastric fluid, which contains not only an acid environment, but also pepsin, the immunoglobulin, IgG is virtually destroyed after one hour. After 2 hours in simulated gastric fluid, there is no IgG detectable. The average time a substance spends in the stomach, or the gastric residence time, averages about two hours. Many times, the gastric residence time can be as long as 5 hours. During this time it is certain that the bulk of the immunoglobulins and other macromolecules contained in colostrum have been destroyed. For example, after 2 hours in simulated gastric fluid, microencapsulated colostrum as described in example 1, still contained about 35% of the IgG in the original sample, whereas, the colostrum that was not microencapsulated (native colostrum), did not contain any IgG after about 30 minutes in the simulated gastric fluid.

In-vivo studies which have been designed to measure the amount of IgG that has survived transit through the stomach and collected at the far end of the small intestine have confirmed the results of in-vitro experiments that were conducted in simulated gastric fluid and simulated intestinal fluid. By the time the colostrum has passed through the stomach and the small intestine, there is very little IGg that can be detected.

It is thought that colostrum, or whey protein, would be most beneficial for gastrointestinal health and for endogenous production of IGF-1, or even direct oral supplementation of IGF-1 itself, if delivered to the small intestine and the colon. To maximize the therapeutic effects of oral IGF-1 mediation, protection of the colostrum or whey from destruction in the stomach is necessary. Colostrum can also be utilized therapeutically for treatment of systemic autoimmune diseases such as lupus. Many clinical studies have been published in the medical literature over the last few years related to the medical benefit of intravenous administration of purified immunoglubulin (IVIGg). If oral colostrum is to be efficacious for this purpose, it must also survive passage through the stomach, and the IGg absorbed into the blood stream from the lower regions of the gastrointestinal tract. The IGg and other macromolecules in the colostrum must remain intact in gastric fluid.

In summary, protection of macromolecules derived from dairy products or recombinant macromolecules such as IGF-1, from the stomach and delivery to the small intestine and the colon is essential for the clinical use of these molecules for the modulation of metabolic and immune function as well as for treatment pathogens that invade the gastrointestinal tract.

In a preferable embodiment, the amount of IGF-1 mediating substance in a single oral dose ranges from about 250 mg. to about 30 grams, and the amount of creatine monohydrate ranges from about 500 mg. to 20 grams. In a more preferable embodiment, the amount of colostrum in a single oral dose ranges from about 500 mg. to 5 grams and the amount of creatine monohydrate ranges from about 2 grams to 10 grams. In a still more preferable embodiment, the amount of whey protein isolate containing 2-5% IgG in a single oral dose would be about 20 grams and the amount of creatine monohydrate in a single oral dose would be about 5 grams.

The preferred formulation of each substance for coadministration would be sustained-release. The same dosage levels of each ingredient that were specified above would be applicable in the sustained-release form. Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems. Physical systems include, but not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., non-erodible, erodible, environmental agent ingression, and degradable), and materials physically dispersed in non-porous, polymeric, or elastomeric matrices (e.g., non-erodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous).

Hydrogels may also be employed as described in “Controlled Release Systems: Fabrication Technology”, Vol. II, Chapter 3; p 41-60; “Gels For Drug Delivery”, Edited By Hsieh, D.

While a preferable mode of sustained-release drug delivery will be oral, other modes of delivery of sustained-release compositions according to this invention may be used. These include mucosal delivery, nasal delivery, ocular delivery, transdermal delivery, parenteral controlled release delivery, vaginal delivery, rectal delivery, and intrauterine delivery.

There are a number of sustained-release drug formulations that are developed preferably for oral administration. These include, but are not limited to, microencapsulated powders, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gel diffusion-controlled gastrointestinal delivery systems; and ion-exchange-controlled gastrointestinal delivery systems, which include cationic and anionic drugs. The preferred sustained-release system is an oil microencapsulated sustained-release powder dosage form that can be mixed with liquid and consumed as a drink beverage.

Combinations of coating agents may also be incorporated such as ethylcellulose and hydroxypropylmethylcellulose, which can be mixed together and sprayed onto the colostrum or whey protein in a fluid bed granulator. Another method employs processing the IGF-1 mediating substance with a high temperature melting vegetable oil with a melting point of about 145° F. This combination can be processed in a vertical or horizontal high intensity mixer or a blender that is jacketed so as to allow a hot water bath to circulate around the mixer to elevate the temperature of the oil to the melting point. The whey or colostrum and creatine powder are then mixed with the molten oil until complete coverage is achieved (about 15-20 minutes), cooled, and discharged. The finished product is a microencapsulated, free-flowing sustained-release powder with an extended release profile.

Examples of carriers useful in solid and aqueous dispersions according to the invention include, but are not limited to, water-soluble polymers such as guar gum, glucommannan, psyllium, gum acacia, polyethylene glycol, polyvinylpyrrolidone, hydroxypropyl methylcellulose, and other cellulose ethers such as methylcellulose, and sodium carboxymethylcellulose. Powdered drink mixes which are designed to be added to water or other liquids incorporating microspheres of sustained-release L-arginine, coated with a high melting point vegetable oil, and then mixed with a hydrocolloid polymer such as those previously listed are also suitable.

Furthermore, compositions of whey, colostrum, or biological equivalents and creatine according to the invention may be administered or coadministered with conventional pharmaceutical binders, excipients and additives. Many of these are controlled-release polymers which must be used in sufficient quantities to produce a sustained-release effect. The use of low levels of these ingredients will not result in sustained-release when they are used as a diluent, binder, or disintegrant. These include, but are not limited to, gelatin, natural sugars such as raw sugar or lactose, lecithin, mucilage, plant gums, pectin's or pectin derivatives, algal polysaccharides, glucomannan, agar and lignin, guar gum, locust bean gum, acacia gum, xanthan gum, carrageenan gum, karaya gum, tragacanth gum, ghatti gum, starches (for example corn starch or amylose), dextran, polyvinyl pyrrolidone, polyvinyl acetate, gum arabic, alginic acid, tylose, talcum, lycopodium, silica gel (for example colloidal), cellulose and cellulose derivatives (for example cellulose ethers, cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate, cross-linked sodium carboxymethylcellulose, cross-linked hydroxypropylcellulose, high-molecular weight hydroxymethylpropycellulose, carboxymethyl-cellulose, low-molecular weight hydroxypropylmethylcellulose medium-viscosity hydroxypropylmethylcellulose hydroxyethylcellulose, hydroxypropylcellulose, hydroxypropylmethylcellulose, sodium carboxymethylcelulose, alkylcelluloses, ethyl cellulose, cellulose acetate, cellulose propionate (lower, medium or higher molecular weight), cellulose acetate propionate, cellulose acetate butyrate, cellulose triacetate, methyl cellulose, hydroxypropyl cellulose, or hydroxypropylmethyl cellulose), fatty acids as well as magnesium, calcium or aluminum salts of fatty acids with 12 to 22 carbon atoms, in particular saturated (for example stearates such as magnesium stearate), polycarboxylic acids, emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈J₃₆O₂ and their mixtures, it being possible for the glycerol hydroxy groups to be totally or also only partly esterified (for example mono-, di- and triglycerides); high melting point hydrogenated vegetable oils suitable for microencapsulation; pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally Also be etherified, esters of citric acid with primary alcohols, acetic acid, urea, benzyl benzoate, dioxolanes, glyceroformals, tetrahydrofurfuryl alcohol, polyglycol ethers with C₁-C₁₂-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other substances that may be used include: cross-linked polyvinyl pyrrolidone, carboxymethylamide, potassium methacrylatedivinylbenzene copolymer, high-molecular weight polyvinylacohols, low-molecular weight polyvinylalcohols, medium-viscosity polyvinylalcohols, polyoxyethyleneglycols, non-cross linked polyvinylpyrrolidone, polyethylene glycol, sodium alginate, galactomannone, carboxypolymethylene, sodium carboxymethyl starch, sodium carboxymethyl cellulose or microcrystalline cellulose; polymerizates as well as copolymerizates of acrylic acid and/or methacrylic acid and/or their esters, such as, but not limited to poly(methyl methacrylate), poly(ethyl methacrylate), poly(butyl methacylate), poly (isobutyl methacrylate), poly(hexyl methacrylate), poly (isodecyl methacrylate), poly(lauryl methacrylate), poly(phenyl methacrylate), poly(methyl acrylate), poly(isopropyl acrylate), poly(isobutyl acrylate), or poly(octadecyl acrylate); copolymerizates of acrylic and methacrylic acid esters with a lower ammonium group content (for example Eudragit® RS, available from Rohm, Somerset, N.J.), copolymerizates of acrylic and methacrylic acid esters and trimethyl ammonium methacrylate (for example Eudragit® RL, available from Rohm, Somerset, N.J.); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethylacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine; poly (ethylene), poly (ethylene) low density, poly (ethylene) high density, poly (propylene), poly (ethylene oxide), poly (ethylene terephthalate), poly (vinyl isobutyl ether), poly (vinyl chloride) or polyurethane. Mixtures of any of the substances or materials listed herein may also be used in the practice of the invention.

Plasticizing agents that may be considered as coating substances useful are: Citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutrate, -dipropionate; tributyl phosphate, tributyrin; polyethylene glycol sorbitan monooleate (polysorbates such as Polysorbar 50); sorbitan monooleate.

Whey, colostrum, or biological equivalent, recombinant IGF-1, and creatine according to the invention may be orally administered or coadministered in a liquid dosage form. For the preparation of solutions or suspensions it is, for example, possible to use water or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of drinkable solutions the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), gum acacia, guar gum, or other suspension agents selected from the hydrocolloids may also be used.

It is also possible to add preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

Furthermore, sustained-release whey or colostrum creatine according to the invention may be administered separately, or may coadministered with other inventive controlled release biological equivalents or other therapeutic agents. Coadministration in the context of this invention is defined to mean the administration of more than the two therapeutic agents in the course of a coordinated treatment to achieve an improved clinical outcome. Such coadministration may also be coextensive, that is, occurring during overlapping periods of time.

It may be preferable at times to administer the creatine in immediate-release form and the whey or colostrum in sustained-release form, or to mix some immediate-release creatine with sustained-release creatine and sustained-release whey to get an initial loading dose of creatine into the system.

The combination of creatine and an IGF-1 stimulating substance such as whey or colostrum, may be used as a food additive or incorporated into a candy bar or other confection like delivery system or food.

Preferred concurrently administered compounds would be selected from the following and may include; L-glutamine, vitamin E, selenium, beta carotene, vitamin C, α-lipoic acid, tocotrienols, N-acetylcysteine, co-enzyme Q-10, Pycnogenol® (French maritime pine bark extract, Henkel, Inc.), extracts of rosemary such as carnosol, botanical anti-oxidants such as green tea polyphenols, grape seed extract, COX inhibitors such as resveratrol, hops (humulus Lupulus L), ginkgo biloba, n-acetyl-cysteine, and garlic extracts. Other amino acid such as L-arginine, L-ornithine, L-cysteine or L-citrulline may be added. Combination with an acetylcholine precurser such as choline chloride or phosphatidylcholine may be desirable to enhance vasodilation.

If an ester or prodrug of either substance is employed, the following could be acceptable; alkyl, ethyl, methyl, propyl, isopropyl, butyl, isobutyl, or t-butyl esters.

If a salt is employed, it could be selected from the following; hydrochloride, glutamate, aspartate, butyrate, or glycolate.

### EXAMPLE 1

Creatine monohydrate and whey protein isolate are blended together and delivered as a powder in a sachet or packet to be mixed in water. Each pre-measured dose contained 5 grams of creatine monohydrate and 10 grams of whey protein isolate containing 3% immunoglobulin A (IgG). Athletes were instructed to consume one packet mixed in water two times per day, for a total daily dose of 20 grams of whey protein isolate and 10 grams of creatine. After 3 weeks of supplementation according to this program, athletes will experience increases in muscle mass when compared with the same dose of creatine without whey protein isolate.

### EXAMPLE 2

Creatine monhydrate and colostrum are added in equal amounts to a jacketed Littleford high intensity mixer which is capable of operating at high temperatures. A hydrogenated soy oil (Dritex S, AC Humko, Memphis, Tenn.) with a melting point of about 80° C. or 140-160° F. was added to the creatine/colostrum powder, and hot water was circulated in the jacket surrounding the vessel so as to raise the core temperature to about 150 degrees F. Mixing was continued for about 20 minutes. Efficient coating or microencapsulation of the powder was achieved when a temperature of about 155° F. was reached and the hot molten oil mixed and coated the powder. The resulting powder was small, free flowing, and exhibited sustained-release properties when a dissolution test was conducted. The weight percent of the finished product was 60% colostrum, 20% creatine, 20% soy oil. This microencapsulated sustained release combination was subjected to in-vitro testing as follows;

Dissolution Testing Protocol

Simulated gastric fluid (SGF) and simulated intestinal fluid (SIF) can be prepared according to USP, or as follows:

1. Preparation of Simulated Gastric Fluid (SGF) (pH 1.2)

Sodium chloride (2 g) and pepsin (3.2 g) are co-dissolved in 7.0 ml of hydrochloride acid. Deionized water is added to make the final volume equal to 1000 ml. pH should be 1.2. Pepsin activity of 800-2500 units per mg. of protein is available from Sigma. Equilibrate to 37 degrees C.

2. Preparation of Simulated Intestinal Fluid (SIF) (pH 7.5)

Monobasic potassium phosphate (23.8 g) is dissolved in 875 ml of water. Sodium hydroxide (665 ml, 0.2N) and 1400 ml of water are then added. Pancreatin (35 g) is added and the resulting solution is adjusted with 0.2N sodium hydroxide to a pH of 7.5+−0.5. The solution is diluted with water to a final volume of 3500 ml. Equilibrate to 37 degrees C.

Use basket method, and set rotation speed at 50 RPM and maintain dissolution media at 37 degrees C.

Sample Points:

30 minutes, 1 and 2 hours in simulated gastric fluid (SGF), drain and refill with SIF.

3, 5 and 8 hours in simulated intestinal fluid (SIF).

SGF Results: % IgG in GI fluids (mg./g)

| Time Point | Native colostrum (mg./g sample) | Micro encapsulated colostrum (mg./g) |
|---|---|---|
| baseline | 200 | 170 |
| 30 minutes | 25 | 55 |
| 1 hour | 0 | 55 |
| 2 hours | 0 | 55 |

SGF-simulated gastric fluid

The formulation of example 2 was then blended with suspending agents, sweetener, and flavor to yield a pleasant tasting powder that could be mixed with water or juice to yield a dose of 5 grams of creatine monohydrate and 5 grams of colostrum with a 20% level of IgG. This formulation produced a sustained-release delivery system designed to increase the storage and delivery of creatine to the muscles, while simultaneously enhancing aerobic capacity. As can be seen from the above data, 35% of the IgG in the microencapsulated colostrum survived up to 2 hours in simulated gastric fluid, whereas, the IgG in the unprotected native colostrum was completely destroyed.

### EXAMPLE 3

Creatine monhydrate is added to a high intensity mixer that is jacketed with a circulating hot water bath which is capable of operating at high temperatures. A vegetable oil with a melting point of about 145 degrees F. is added and mixing is commenced. While mixing, the temperature is elevated to about 160° F. until complete melting of the oil is achieved, and the contents are well mixed. Efficient coating or microencapsulation of the powder was achieved in about 20 minutes when the hot oil throughly mixed with the powder. The mixer was than cooled by running cool water through the jacketed system surrounding the unit, and the resulting free flowing powder discharged. The powder particles were small, free flowing, and exhibited sustained-release properties when a dissolution test was conducted. The weight percent of the finished product was 2% vegetable oil, and 98% creatine. Sustained release was verified by the following dissolution test;

Dissolution Test

| | |
|---|---|
| Protocol: | |
| Basket method | Paddle speed: 50 RPM |
| Media: water | Time points: 1, 2, 4, 6, and 8 hours |
| Results: % release | |
| Creatine | |

| | |
|---|---|
| 1 hr | 46.5% |
| 2 hrs | 63.6% |
| 4 hrs | 78.6% |
| 6 hrs | 84.9% |

The formulation of example 3 can be blended with whey protein isolate, or preferably, with a whey protein isolate that has been microencapsulated to further protect the inherent macromolecules

The formulation of the above examples can be tested in athletes and body builders and shown to increase muscle mass and contribute to sustained or prolonged endurance. This formulation will be shown to be particularly effective in sports activities of long duration. In addition, the formulations of the above examples will be shown to significantly enhance or increase creatine transport or creatine loading when compared with creatine alone. This is an unexpected result, which has heretofore not been suggested or demonstrated in animals or humans as far as the inventor is aware. Creatinine and creatine kinase are additional biochemical markers that can be measured in blood or as metabolites in urine to further measure creatine transport, metabolsim, or modulation in muscle. Differences between creatine alone and creatine in combination with an IGF-1 mediating substance such as whey or colostrum will be noticable by looking at creatine metabolism.

While the present invention is described above in connection with the preferred or illustrative embodiments, those embodiments are not intended to be exhaustive or limiting of the invention, but rather, the invention is intended to cover any alternatives, modifications or equivalents that may be included within its scope as defined by the appended claims.

What is claimed is:

1. An oral creatine transport enhancing composition comprising:
a. creatine or a pharmaceutically acceptable salt, ester, polypeptide, complex, prodrug, metabolite, or derivative of creatine; and
b. whey protein, colostrum, or human recombinant IGF-1.

2. The composition of claim 1, wherein the creatine is creatine monohydrate, carnitine creatinate, creatine pyruvate, or cyclocreatine.

3. The composition of claim 1, wherein the whey protein is whey protein isolate or whey protein concentrate

4. The composition of claim 3, wherein the creatine is creatine monohydrate in an amount between about 500 mg and about 20 grams and the whey protein isolate is in an amount between about 500 mg and about 30 grams.

5. The composition of claim 1, wherein the creatine monohydrate and whey protein, colostrum, or IGF-1 are sustained-release, micro encapsulated, or enteric coated.

6. The composition of claim 1, wherein the whey protein or colostrum is sufficient to mediate IGF-1.

7. The composition of claim 3, wherein the whey protein isolate or concentrate is microencapsulated to be sustained release or enteric coated.

8. A composition of claim 5 wherein the sustained-release agent comprises algal polysaccharides, chitosan, pectin, glucomannan, guar gum, xanthan gum, gum arabic, gum karaya, locust bean gum, keratin, laminaran, carrageenan, cellulose, modified cellulosic substances such as cellulose ether derivatives; methylcellulose, hydroxypropylmethylcellulose, hydroxypropylcellulose, hydroxyethylcellulose, sodiumcarboxymethylcellulose, carboxymethylcellulose carboxypolymethylene, acrylic resin polymers, polyacrylic acid and homologues, polyethylene glycol, polyethylene oxide, polyhydroxylalkyl methacrylate, polyvinylpyrollidine, polyacrylamide, agar, zein, stearic acid, high melting point oils, waxes, gelatin, or combinations thereof.

9. A composition of claim 8 wherein the sustained-release particles are produced by coating with an oil with a melting point greater than 100° F.

10. A composition of claim 8 wherein the oil is a vegetable oil with a melting point of approximately 150° F.

11. A method of enhancing creatine transport to muscle in a mammal comprising the steps of administration of creatine or a pharmaceutically acceptable salt, ester, polypeptide, complex, prodrug, metabolite, or derivative of creatine; and coadministration of whey protein, colostrum, or human recombinant IGF-1.

12. The method of claim 11 wherein both substances, or one or the other is sustained-release.

13. The method of claim 12, wherein the creatine is creatine monohydrate in an amount between about 500 mg and about 20 grams and the whey protein or colostrum is in an amount between about 500 mg and about 30 grams.

14. The method of claim 13, wherein the composition comprises whey protein isolate or colostrum together or separately and creatine monohydrate and wherein any one or all of the components are sustained-release or delayed release.

15. A method of enhancing creatine transport to muscle in a mammal comprising the steps of administration of creatine or a pharmaceutically acceptable salt, ester, polypeptide, complex, prodrug, metabolite, or derivative of creatine; and coadministration of whey protein, recombinant IGF-1, or colostrum.

16. The method of claim 15, wherein the creatine is creatine monohydrate in an amount between about 500 mg and about 20 grams and the amount of whey protein or colostrum is in an amount between about 500 mg and about 30 grams.

17. The method of claim 16, wherein the creatine monohydrate and the whey protein or colostrum are sustained-release or enteric coated.

18. A method of increasing muscle mass in humans comprising the steps of administration of creatine or a pharmaceutically acceptable salt, ester, polypeptide, complex, prodrug, metabolite, or derivative of creatine; and coadministration of whey protein isolate or colostrum.
